(19) United States
(12) Patent Application Publication
(10) Pub. No.: US 2014/0371566 A1
(43) Pub. Date: Dec. 18, 2014

(54) CONFORMING PATIENT CONTACT INTERFACE AND METHOD FOR USING SAME

(71) Applicant: CardioThrive, Inc., Concord, CA (US)

(72) Inventors: Douglas M. RAYMOND, Livermore, CA (US); Peter D. GRAY, Vallejo, CA (US)

(21) Appl. No.: 14/303,538

(22) Filed: Jun. 12, 2014

Related U.S. Application Data
(60) Provisional application No. 61/835,433, filed on Jun. 14, 2013.
(60) Provisional application No. 61/835,435, filed on Jun. 14, 2013.

(51) Int. Cl. A61B 5/0408; A61N 1/04

(52) U.S. Cl. CPC A61B 5/0408; A61N 1/0488
USPC 600/386; 607/142

(57) ABSTRACT

A conforming patient contact interface and method of using the same are disclosed. The conforming patient contact interface may flex when deployed to better contact a surface of a body of a patient.

### PRIORITY CLAIMS/RELATED APPLICATIONS

This application claims the benefit and priority under 35 USC 119(e) and 120 to U.S. Provisional Patent Application Nos. 61/835,433, filed on Jun. 14, 2013 and titled “Conforming Sensor and Method For Using Same” and 61/835,435, filed on Jun. 14, 2013 and titled “Conforming Electrode and Method For Using Same”, the entirety of both of which are incorporated herein by reference.

### FIELD

The disclosure relates generally to methods and arrangements relating to medical devices or consumer devices and, more specifically, to the patient contact interfaces used in medical devices. In one implementation, the disclosure relates to patient contact interfaces used in an external defibrillator.

### BACKGROUND

A primary task of the heart is to pump oxygenated, nutrient-rich blood throughout the body. Electrical impulses generated by a portion of the heart regulate the pumping cycle. When the electrical impulses follow a regular and consistent pattern, the heart functions normally and the pumping of blood is optimized. When the electrical impulses of the heart are disrupted (i.e., cardiac arrhythmia), this pattern of electrical impulses becomes chaotic or overly rapid, and a sudden cardiac arrest may take place, which inhibits the circulation of blood. As a result, the brain and other critical organs are deprived of nutrients and oxygen. A person experiencing sudden cardiac arrest may suddenly lose consciousness and die shortly thereafter if left untreated.

The most successful therapy for sudden cardiac arrest is prompt and appropriate defibrillation. A defibrillator uses electrical shocks to restore the proper functioning of the heart. A crucial component of the success or failure of defibrillation, however, is time. Ideally, a victim should be defibrillated immediately upon suffering a sudden cardiac arrest, as the victim's chances of survival dwindle rapidly for every minute without treatment.

There are a wide variety of defibrillators. For example, implantable cardioverter-defibrillators (ICD) involve surgically implanting wire coils and a generator device within a person. ICDs are typically for people at high risk for a cardiac arrhythmia. When a cardiac arrhythmia is detected, a current is automatically passed through the heart of the user with little or no intervention by a third party.

Another, more common type of defibrillator is the automated external defibrillator (AED). Rather than being implanted, the AED is an external device used by a third party to resuscitate a person who has suffered from sudden cardiac arrest. FIG. 8 illustrates a conventional AED 800, which includes a base unit 802 and two pads 804. Sometimes paddles with handles are used instead of the pads 804. The pads 804 are connected to the base unit 802 using electrical cables 806.

A typical protocol for using the AED 800 is as follows. Initially, the person who has suffered from sudden cardiac arrest is placed on the floor. Clothing is removed to reveal the person's chest 808. The pads 804 are applied to appropriate locations on the chest 808, as illustrated in FIG. 8. The electrical system within the base unit 800 generates a high voltage between the two pads 804, which delivers an electrical shock to the person. Ideally, the shock restores a normal cardiac rhythm. In some cases, multiple shocks are required.

Some current types of External Defibrillators, whether they are manual, semi-automatic or automatic versions, make use of rigid paddles for delivering therapeutic shocks to a patient and such types are all constrained by the rigid flat paddle bases. These rigid flat paddle bases do not conform to the curvatures of the patient's body at the locations on the body where the paddles must be placed to be most effective. As such, the operators of these devices must apply a good amount of contact force to maximize the physical contact across the entire paddle sensor/electrical interface and to maintain this force for the entire time that it takes the defibrillator to clearly and unambiguously sense the heart's rhythm. The operator must also continue holding the paddles in place and to maintain the required contact force while the device analyzes the patient's ECG, and in the case of an automatic or semi-automatic unit decides upon the appropriate course of action (shock or no shock), and then delivers the therapeutic action. The need for the operator to hold the paddles in place increases the risk of poor contact with the patient resulting in the potential misreading of the heart rhythm, or in failing to appropriately deliver the required therapeutic action. In addition, the operator is at some risk of being shocked by the defibrillation pulse and hence at risk of physical harm or death.

Some times in place of the rigid paddles, external defibrillators utilize flexible electrode pads made of foam and conductive foil. However, such electrode pads are fragile and easily damaged and hence currently they have to be protected and separately packaged and stored within the hard outer shell of automated external defibrillators, requiring the operator to find them, unpack them and then connect them to the main defibrillator body before they are ready to be used on the patient. Thus, this significant amount of time delays unnecessarily the delivery of the therapeutic shock to the patient.

### BRIEF DESCRIPTION OF THE DRAWINGS

FIG. 1 illustrates a side of a conforming patient contact interface, which goes in contact with the patient that is in an inactive or undeployed state.

FIG. 2 illustrates a side of the conforming patient contact interface opposite to the one shown in FIG. 1 that is in an inactive or undeployed state.

FIG. 3 illustrates the conforming patient contact interfaces shown in FIGS. 1 and 2 in a flexed state.

FIG. 4 illustrates the conforming patient contact interface in an active or deployed state with different levels of flexure.

FIG. 5 illustrates the conforming patient contact interface in an active or deployed state in which each patient contact interface element may be independently moved.

FIG. 6 illustrates an embodiment of the conforming patient contact interface in a deployed state where the conforming patient contact interface is part of a treatment electrode within a flat surface of a rigid external defibrillator paddle.

FIG. 7 illustrates via a cross-section view of the conforming patient contact interface in an active or deployed state once applied to the non-flat surface of a patient's body surface.

FIG. 8 diagrammatically illustrates an example of a conventional defibrillator.

### DETAILED DESCRIPTION OF ONE OR MORE EMBODIMENTS

The disclosure is particularly applicable to an external defibrillator that has conforming patient contact interfaces and it is in this context that the disclosure will be described. It will be appreciated, however, that the device and techniques described below has greater utility since the conforming patient contact interfaces may be manufactured differently than described below and the conforming patient contact interfaces may be used with any consumer device or medical device in which it is desirable to have a conforming patient contact interface that may, for example, conform to a patient.

In one implementation described below, the conforming patient contact interfaces allow external defibrillators with rigid paddles, or any other type of medical device with rigid patient contact surfaces, to have the patient contact surface of the paddles (or other medical device surface) conform to the bodily curves of the patient. The conforming patient contact interfaces thus improve the levels of physical contact with the patient without the need to apply excessive contact force.

In the one implementation described below, the conforming patient contact interfaces also allow external defibrillators, or other medical devices with patient contact surfaces, to be positioned on a person or patient and then left alone since the conforming patient contact interface ensures that the sensor and electrode element surfaces remain in optimal contact with the patient. For the external defibrillators, the conforming patient contact interfaces ensure that the person who is being treated by the defibrillator (the victim or the wearer) can have both high quality sensor-to-patient connections and high quality electrode-to-patient connections and hence receive the full benefit of the therapeutic shock. Currently many medical devices are positioned on a person or patient and the operator then has to remain holding them in place until the treatments have been finished. This usually requires that the operator has a high level of medical training or at least a high level of training with the specific medical device concerned.

The conforming patient contact interfaces may also use additional adhesive on the edges of the rigid patient contact surfaces that helps to hold the device in place while the flexibility of the conforming patient contact interfaces contacts the patient body, and conforms its shape to that of the patient's body, eliminating any further need for the operator to be in contact with the medical device or the patient, and thus reducing the risk to the patient. This frees up the operator to perform other tasks or to focus more fully on the readings generated by the device. This also reduces the risk of the operator introducing additional noise or artifacts to the sensors' readings or from interfering with the successful delivery of any therapeutic shock to the patient or of receiving an accidental shock himself or herself.

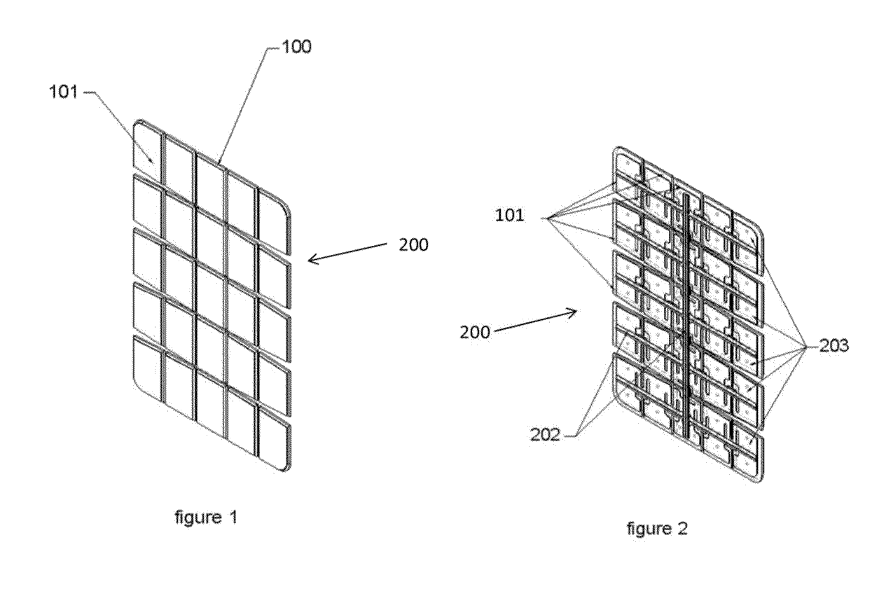

FIG. 1 illustrates a side 100 of a conforming patient contact interface, such as a treatment electrode, which goes in contact with a patient that is an inactive or undeployed state. A second side 200 of the conforming patient contact interface is also shown and the conforming patient contact interface has a thickness that separates the first and second sides of the conforming patient contact interface. The side 100 may include a set of patient contact elements 101 separated by gaps as shown. Each set of patient contact elements 101 may vary in shape and number to suit the exact need for the particular device in which the patient contact interface is being used and hence to provide the best results. The surfaces of the patient contact elements 101 may consist of electrically conductive surfaces, or may instead be covered with sensors or adhesive or other substances or devices useful to the purpose of the medical device involved. The one or more gaps in the side 100 allow the side to bend and flex so that it may, for example, conform to a surface of a patient on which the conforming patient contact interface is placed.

The patient contact elements 101, 102 may be one or more sensors, one or more electrodes or a combination of one or more sensors and one or more electrodes. In some implementations in which the patent interface assembly has both sensors and electrodes, the sensors and electrodes may each be located separately from each other. In other implementations in which the patent interface assembly has both sensors and electrodes, the sensors and electrodes may be intermixed with each other in the patient interface assembly.

The patient interface assembly described in this document may be placed onto a body of a patient and may be used, for example, to sense the heartbeat of the patient and then deliver a therapeutic pulse to the patient for defibrillation for example. The patient interface assembly may also be used to deliver other types of treatments of varying during to the patient. The patient interface assembly may also be used to sense a characteristic of the patient, such as a heartbeat or pulse and the like. The patient interface assembly may also be used to both sense a characteristic of the patient and deliver a treatment to the patient when the patient interface assembly has both sensors and electrodes.

The patient contact assembly may be placed onto the body of the patient at various locations, such as the torso, limbs and/or head of the patient. In some implementations, multiple patient contact assemblies may be used and each patient contact assembly may be placed on one or more locations on the body of the patient. In some embodiments, the patient contact assembly may have one or more patient contacts 101, 102 as shown in FIG. 1 and each patient contact may have the same particular shape (which is not shown in FIG. 1.) In other implementations, the patient contact assembly may have one or more patient contacts 101, 102 as shown in FIG. 1 and each patient contact may have a variety of shapes such as those shown in FIG. 1 for example. Similarly, each patient contact may be similarly sized or differently sized as shown in FIG. 1.

FIG. 2 illustrates the side 200 of the conforming patient contact interface, which is opposite to the one in contact with the patient and is shown in an inactive or undeployed state. The patient contact elements 101 of the first side 100 may be connected, on the side 200, through a series of conductive material plated through-holes in the patient contact elements 101 and soldered, or otherwise connected via an electrically conductive pathway such as a flexible interconnecting circuit 203 as shown in FIG. 2. The electrically conductive pathway allows electrical signals to be communicated to/from each patient contact element 101, such as supplying power or an interrogating signal to each patient contact element 101 and receiving signals from each patient contact element 101.

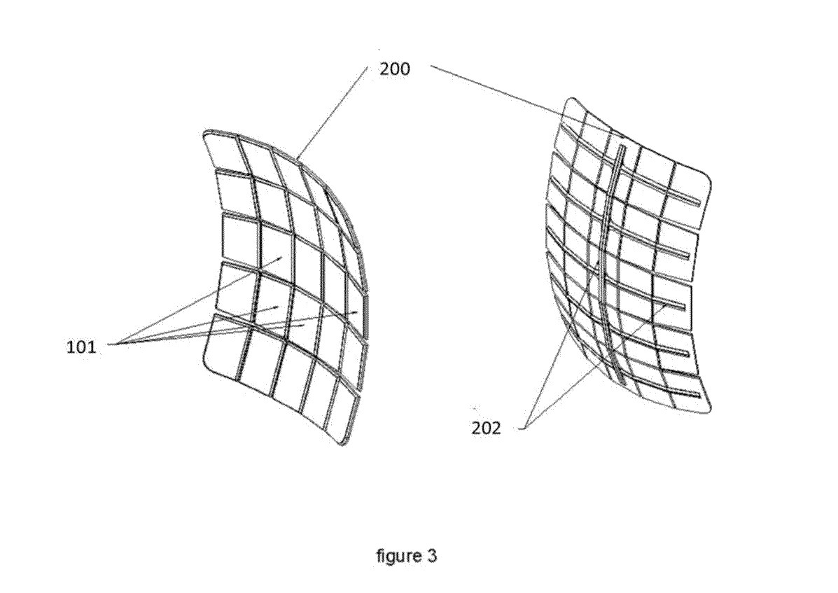

The combination of the first and second sides 100, 200 shown in FIGS. 1-2 may form the patient contact element assembly. The patient contact element assembly may be arranged in such a manner as shown in FIG. 3, but the patient contact elements 101 can also be interspersed with sensors which can be used to detect much more sensitive signals through the appropriate use of signal processing to identify and remove noise from the signals that are wanted. The types of sensors that can be found may include, but are not limited to an ECG sensor, a pulse sensor, a temperature sensor, a blood oxygenation sensor, strain gauges, a skin conductivity sensor, a moisture sensor, an accelerometer or a microphone. In the case of ECG sensors, those sensors can more accurately detect and remove the artifacts created by a patient's breathing or gasping, muscle movements, external vibrations or motion or even from external electromagnetic signals. The mix of sensor types may further include sensors, which can be active in nature, passive in nature, or a combination of the two types. A passive sensor may be a sensor, like an ECG sensor, that just passively picks up a reading or signal, without taking any action itself. An active sensor is a sensor, such as a Pulse Oximeter, that actively performs a function such as shining a light into the patient's flesh in order to detect and analyze the reflected light from the blood flow in the patient's nearby blood vessels and hence identify the levels of oxygenation of that blood.

Furthermore, a particular patient contact element 101 can also be used for non-sensor and non-treatment purposes. For example, the patient contact element may hold adhesive or some other type of mechanical device. The gaps in the side 100 may have one or more strain gauges between the patient contact elements 101 rather than being located solely on the patient contact elements themselves.

The second side 200 may also have a longitudinal linear spring spine with radiating arms 202 runs along the center of the assembly and the arms radiate out perpendicular causing the portions of the one or more flexible interconnecting circuits 203 to which it is attached, and the connected patient contact elements 101 to curve up on the ends of the patient contact assembly 100, as shown in FIG. 3 when in an active or deployed state. The patient contact elements 101 may be flat when in an inactive or undeployed state. This shape change actuation of the spine 202 may be accomplished by mechanical, electrical or other means, such as by using a material that is mechanically responsive to an electrical current, or a material that is under mechanical tension or compression when laying flat and that automatically returns to its resting state in the arced shape, or else through another equivalent means. The material for this component may include, but is not limited to: Stainless Steel; Chrome Silicon; Chrome Vanadium; Phosphor Bronze and suitable bimetallic combinations. Alternatively to the longitudinal linear spring spine with radiating arms in FIG. 2, other forms of actuation or articulation may be use and may be connected to the patient contact elements either individually or in groups. The use of a longitudinal linear spring spine or its equivalent gives the patient contact interface a wide range of possible axes of movement for each patient contact element or for each patient contact element array.

The second side 200 of the patient contact element assembly may also have one or more actuators coupled to the second side. The one or more actuators may be either passive actuators or active actuators or a combination of active actuators and passive actuators. The one or more actuators may positions each of the one or more patient contact elements in the exact manner to better conform to a body surface of a patient. Each of the active actuators may be processor controlled and may include suitable sensors for an active feedback and positioning mechanism.

In one embodiment, the patient contact interface allows external defibrillators with rigid paddles, or any other type of medical device with rigid sensor surfaces, to have the patient contact surface of the paddles (or other medical device surface) conform to the bodily curves of the patient, thus improving the levels of physical contact without the need to apply excessive contact force. Furthermore, the patient contact assembly allows patient contact interfaces to be positioned on a person or patient and then left alone and in place as the patient contact assembly ensures that the patient contact surfaces remain in optimal contact with the wearer and hence ensure that the therapeutic treatment occurs as intended.

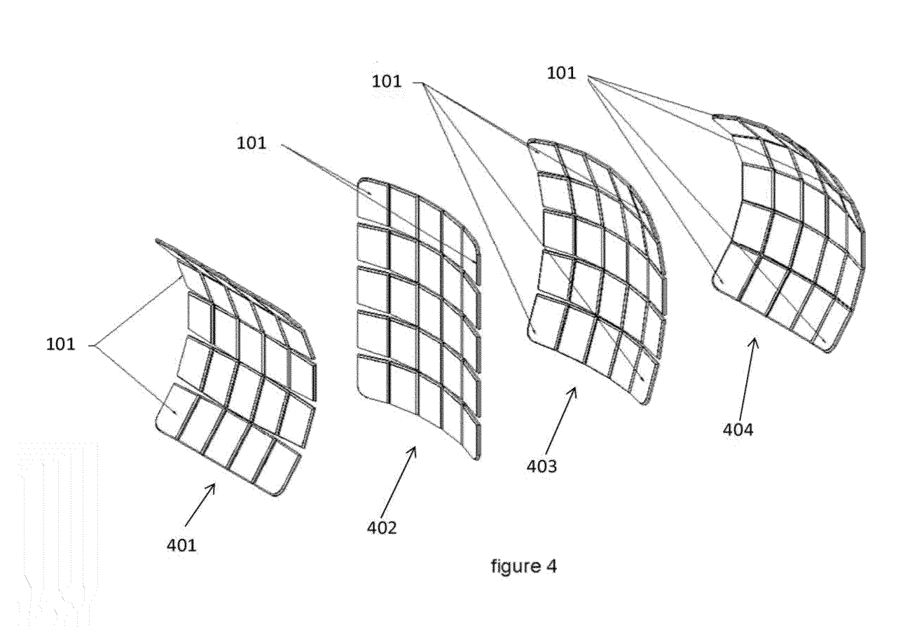

FIG. 4 shows the patient contact assembly 100 in a series of flexed positions. In the example in FIG. 4, the patient contact assembly 100 may be rectangular shape with a first shorter side and a longer second side. For example, in a first position 401, the patient contact assembly 100 is shown where the longitudinal linear spring with radiating arms 202 (not shown here) has the arm springs not flexed but the spine spring is at full flexure so that the patient contact elements 101 are flexed along the longer second side at the top and bottom edge of the rectangular array in the example in FIG. 4. In position 402, the longitudinal linear spring with radiating arms 202 (not shown here) of the patient contact assembly 100 may have a longer spine spring (along the middle of the longer side of the rectangle) not flexed, but the arm springs at full flexure so that the patient contact elements 101 at each end of the first shorter side may be flexed.

In position 403, the longitudinal linear spring with radiating arms 202 (not shown here) of the patient contact assembly 100 may have both the spine spring and arm springs partially flexed. In position 404, the longitudinal linear spring with radiating arms 202 (not shown here) of the patient contact assembly 100 may have the arm and spine springs both at full flexure. Due to these different flexed positions, the patient contact elements 101 are free to conform to the surface of the patient, and are held against the patient's skin by the force of the longitudinal linear spring with radiating arms 202.

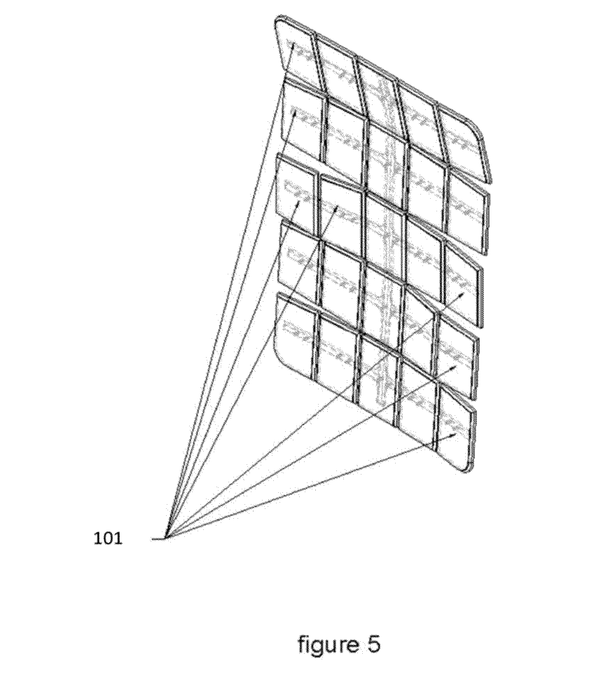

FIG. 5 shows how the conforming patient contact assembly 100 allows each of the patient contact elements 101 to move independently from each other, thus providing optimal contact with the patient's skin surface. This maximizes the contact efficiency needed for providing effective diagnosis of, and therapeutic action to, the patient.

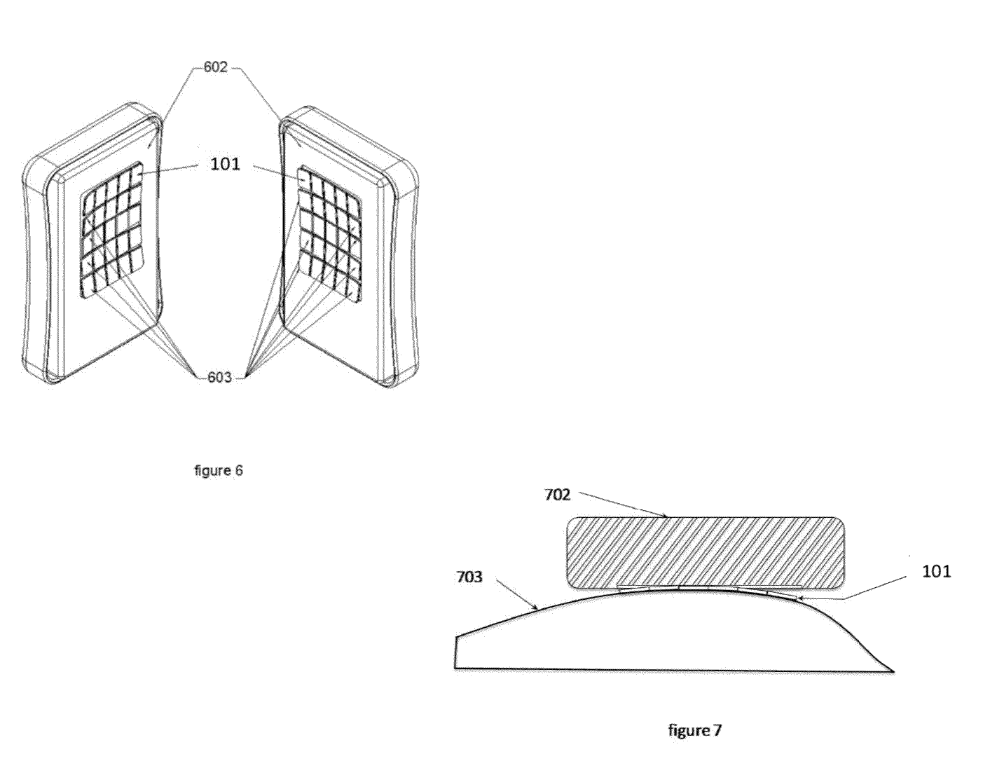

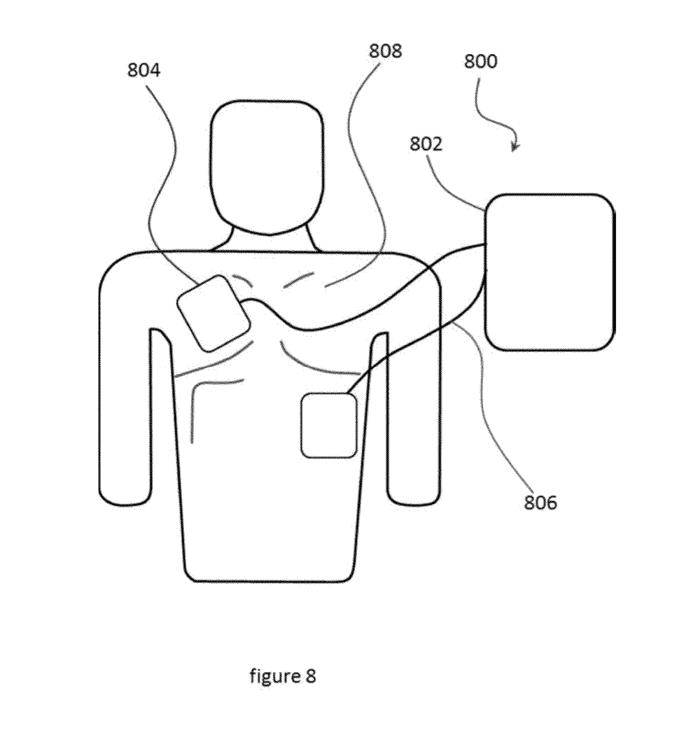

FIG. 6 illustrates an embodiment of the conforming patient contact interface in a deployed state where it is in use as an electrode within the flat surface of a rigid external defibrillator paddle 602. Specifically, an external defibrillator or defibrillator paddle surface 602 has one or more patient contact element assemblies 101 that may be anchored to the defibrillator paddle body 602 at the center point of the assembly, as shown in FIG. 6. As before, the flexibility of the flexible interconnecting circuit allows for multiple axes of movement for the patient contact elements 101 ensuring an optimal contact surface 603 with the surface of the body of the patient.

FIG. 7 illustrates via a cross-section view of the patient contact assembly in an active or deployed state once applied to the non-flat surface of a patient's body surface. In FIG. 7, a conforming patient contact interface 101 is in contact with a curved surface 703 that represents a patient's body, while attached to the rigid external defibrillator paddle 702. Here can be seen the ability for the conforming patient contact interface 101 to place all patient contact interface surfaces in contact with the patient's skin 703 even when the device has a rigid paddle 702.

While the foregoing has been with reference to a particular embodiment of the invention, it will be appreciated by those skilled in the art that changes in this embodiment may be made without departing from the principles and spirit of the disclosure, the scope of which is defined by the appended claims.

1. A conforming patient contact, comprising:
a first side having one or more patient contact elements; and a second side, opposite the first side, having one or more conductive traces that make electrical contact with the one or more patient contact elements so that patient contact conforms to a body surface of a patient when the patient contact is placed on the body surface of the patient.

2. The patient contact of claim 1 further comprising a spine, coupled to a surface of the second side, that flexes the one or more patient contact elements in an arc to better conform to a body surface of a patient.

3. The patient contact of claim 2, wherein the spine further comprises a spine spring running down a center region of the second side and one or more spring arms that are perpendicular and connected to the spine spring wherein the spine spring and the one or more spring arms flex in different directions.

4. The patient contact of claim 1, wherein the second side further comprises a plurality of actuators coupled to the second side, wherein each actuator positions each of the one or more patient contact elements to better conform to a body surface of a patient.

5. The patient contact of claim 4, wherein each actuator is one of a passive actuator and an active actuator.

6. The patient contact of claim 5, wherein the active actuator further comprises a processor and a sensor wherein the processor controls the actuation of the actuator and the sensor provides active feedback to the processor of the actuation of the actuator.

7. The patient contact of claim 1, wherein the patient contact is placed on a body of a patient.

8. The patient contact of claim 7, wherein the body of the patient is one of a torso of the patient, a limb of the patient and a head of the patient.

9. The patient contact of claim 1, wherein each of the one or more patient contact elements has a predetermined size.

10. The patient contact of claim 1, wherein at least one of the one or more patient contact elements has a different size.

11. The patient contact of claim 1, wherein at least one of the patient contact elements is a sensor.

12. The patient contact of claim 11, wherein the sensor is one of an active sensor and a passive sensor.

13. The patient contact of claim 1, wherein the patient contact elements further comprise at least one active sensor and at least one passive sensor.

14. The patient contact of claim 1, wherein at least one of the patient contact elements is an electrode.

15. The patient contact of claim 14, wherein the patient contact elements further comprise at least two electrodes and at least two sensors.

16. The patient contact of claim 15, wherein the electrodes and the sensors are one of grouped separately on the first side and intermixed on the first side.

17. The patient contact of claim 1, wherein each of the one or more patient contact elements are spaced apart from each other to allow the conforming patient contact to flex and conform to a contour of the body surface.

18. A method for attaching a patient contact to a patient, the method comprising:
providing a patient contact having a first side having one or more patient contact elements and a second side, opposite the first side, having one or more conductive traces that make electrical contact with the one or more patient contact elements; and
placing the patient contact on a body of a patient.

19. The method of claim 18, wherein placing the patient contact further comprises placing one or more patient contacts at a location on the body of the patient.

20. The method of claim 19, wherein the location on the body of the patient is one of a torso of the patient, a limb of the patient and a head of the patient.

21. The method of claim 18 further comprising delivering, using the patient contact, a treatment to the patient.
